(19) United States
(12) Patent Application Publication
(10) Pub. No.: US 2006/0002995 A1
(43) Pub. Date: Jan. 5, 2006

(54) Pharmaceutical porous particles

(76) Inventor: Ian Harwigsson, Malmo (SE)

Correspondence Address: Matthew E. Connors;Gauthier & Connors LLP, Suite 2300, 225 Franklin Street, Boston, MA, 02110, US

(21) Appl. No.: 11/149,927

(22) Filed: Jun. 10, 2005

Related U.S. Application Data
(63) Continuation of application No. PCT/SE03/01952, filed on Dec. 15, 2003.

(30) Foreign Application Priority Data
Dec. 13, 2002 (SE) 0203687-9

(51) Int. Cl. A61K 9/127

(52) U.S. Cl. 424/450

(57) ABSTRACT

The present invention relates to a pharmaceutical, preferably inhalable, porous, free flowing particle to be used in therapeutical application, optionally comprising a therapeutically active compound or substance, whereby the particle consists of one or more network forming compounds, which in diluted solutions self associates to large three dimensional structures having a density of <0.5 g/cm³.

### TECHNICAL FIELD

The present invention relates to solid particles to be used in therapeutical applications, e.g., as pharmaceuticals or as carriers of drugs as well as a process for their manufacture. This will also include the use thereof as a technical means e.g., at the development of inhalators. The particles are to be used by animals as well as by humans. The particles are particularly designed for inhalation therapy.

As a carrier the active compound or substance may either be mixed into the particles or be attached to the surface of the particles. The active compound can be in any form, e.g., molecularly mixed or in form of particles. The particles have a low density and may be produced using constituent bodily substance(-s). In particular constituent bodily lipids.

### BACKGROUND OF THE INVENTION

A concept, which in recent days has obtained great attention, is the administration of drugs via the airways, in particular the lung. Today, this market is dominated by drugs directed towards diseases in the airways, such as asthma, cystic fibrosis, and Chronic Obstructive Pulmonary Disease (COPD), but systemic administration of drugs via the respiratory routes is expected to increase strongly. There are several advantages using systemic administration via the respiratory airways. For example this administration route, compared to oral administration, avoids decomposition in the gastrointestinal tract as well as in the liver. This together with the, sometimes great, difference in permeability leads to a higher bioavailability. For biomolecules the difference in bioavailability between airway and oral administration may be as large as a factor of 100 or more. The increased bioavailability leads to smaller doses into the body, which in turn may reduce side effects. In the case of expensive active compounds this fact will of course also reduce the production cost of the pharmaceutical; Normally administration via the airways will also result in a more rapid uptake of the active compound relative to other administration routes, with the exception of intravenous injections. Intravenous injections may, however, only be carried out at hospitals while at the same time most patients experience inhalation of a drug as considerably more convenient than injection. The concept to administer drugs systemically via the airways opens up completely new treatment possibilities and is well accepted from a medical point of view. Several pharmaceutical companies have ongoing clinical studies in late phase including inter alias insulin (Ogden 1996).

Today, GlaxoSmithKline has a nasal spray on the market for the systemic administration of sumatriptan (Imigran™) against migraine.

Vaccination and other immunization via the airways/lungs has also been proposed and tested.

The great advantages and the potential of administration via the pulmonary route have lead to that this area has developed rapidly by several developing companies and manufacturers.

Lipids are well suited to be part of compositions for administration via the airways for several reasons. From a general point of view lipids are chemically inert and are thereby well suited to be used as additives to therapeutical compounds. Furthermore, several lipids are constituent bodily substances and are also present in the airways. Further, there is support for the fact that lipids, after deposition in the lung, are spread onto the lung surface and thereby also have the ability of spreading any active compound over a larger surface (Rless, Schutt et al. 2001). Lipids are also added to improve the disaggregating properties of powders (Hanes, Edwards et al. 1999) and to reduce irritation of the pulmonary mucosa (the mucosa of the lung), (Reul and Petrl 1997). Other advantages that one may obtain are a reduced phagocytosis, an increased uptake, and a possibility of varying the time of uptake, (Bhat, Cuff et al. 2000).

Lung lipids, as such, are added to the lung at different disease conditions, such as at IRDS (Infant Respiratory Distress Syndrome) and ARDS (Adult Respiratory Distress Syndrome).

Furthermore, lung lipids have turned out to influence the transport of mucus in the airways. Treatment using suitable lipids may reduce the viscosity of the mucus and may thereby improve the ability to the patient to reduce the amount of mucus in the lung by means of an increased out transport (Pruss 1997). For this reason it has been proposed to medicate with lipids when treating COPD (Riess, Schutt et al. 2001). Studies indicate that lipids can bind to the tissue surface of the lungs, thereby reducing the number of receptors exposed to noxious stimuli and reducing the broncho-constrictor reflex. (Hills, Woodcock et al. 2000)

It is often more complicated to produce pharmaceuticals to be administered via the lung than to produce a common tablet. One of the difficulties is that the particle must have an aerodynamic diameter that is 0.5-5 μm to obtain a satisfactory deposition of the pharmaceutical in the lung. Larger particles (>10 μm) deposit in the oral cavity and the pharyngeal region while smaller particles (<0.1 μm) accompany the expiration air out again.

The most common way of administering inhalable particles is to use dosage aerosols. The active compound is thereby dissolved or suspended in a liquid having a high vapour pressure, usually halogenated hydrocarbons. By means of natural pressure the liquid is pressed through a nozzle at a high speed and forms droplets which evaporate in the oral cavity to form particles of suitable size. Another alternative is to use nebulization where the active compound is dissolved or suspended in an isotonic aqueous solution which is finely distributed into small droplets in a nebulizing apparatus using compressed air or a piezoelectric crystal.

The weaknesses using these administration ways are several. A dosage aerosol utilizes poorly the total amount of a pharmaceutical as only a lesser amount of the droplets will obtain an aerodynamically correct size to be deposed in the lung. The high velocity of the droplets further contributes to the fact that part of the dosage fastens in the rear of the pharyngeal region and gives raise to inconvenience, so called scold freon effect. Restriction for use of certain halogenated hydrocarbons together with the difficulty the pharmaceutical industry has of finding alternative propellants creates furthermore large question marks concerning the future use of dosage aerosols.

At nebulization the administration of one dose normally requires several minutes and requires expensive and cumbersome equipment, which is inconvenient to the patient to use and to bring with.

Manufacture of stable suspension raises great requirements on the formulation which has to have a very low solubility of the suspended agent simultaneously as the tendency of flocculation, precipitation, and aggregation have to be minimized. Solutions on the other hand often give raise to stabilisation problems, particularly in nebulization products where aqueous solutions are used and the active compound is sensitive to hydrolysis, as e.g. proteins and peptides.

The above drawbacks are reduced by administering the pharmaceutical using a dry powder inhalator (DPI). Particles having a suitable aerodynamic size comprising the active compound is dosed using a simple device and is administered to the lung using one or a few inhalations per dosage. No propellant is thereby needed and the stability problems are minimized mainly due to the dry state. As the freedom of choosing excipients is considerably larger than when it comes to suspensions a stability improving composition may be more easily formulated.

Administration of solid particles within this size range using a dry powder inhalator means challenges when it comes to dosage and disaggregating. At inhalation quite often small amounts are dosed which requires a good dosage equipment.

Traditionally, solid inhalable particles are produced by means of a relatively compilcated grinding process or by means of spray drying. Particles within the size range of interest (0.5-5 μm, at the density=1 g/cm³) have normally bad flow properties which makes it difficult to handle. In order to be able to handle small particles one normally has to make them free flowing. Two common ways to obtain free flowing properties is spheronization and ordered mixtures. When spheronizing one creates larger, somewhat loosely adhering agglomerates of the small inhalable particles. In ordered mixtures the inhalable particles are attached onto larger carrier particles, often lactose. These carrier particles are most often considerably larger than the drug particles, in the size of 20-200 μm. In the inhalator the inhalable particles are once again “released” by means of the shearing forces which are obtained when the patient inhales through the inhalator.

At Inhalation, disaggregating of the powder into small particles is a necessity for high dose deposition into the lung. Much work has been done to prevent aggregation, e.g., by trying to control the surface properties of the particles. Additions of so called “force control agents” (FCA), e.g., magnesium stearate, to the formulations has shown to provide for an increased disaggregating, at inhalation, as well. (Staniforth 1996)

In later years, the alternative of making inhalation particles having a low density started to become commercialized (Edwards, Langer et al. 1999; edwards, Caponetti et al. 1999; tarara, Weers et al. 2000; edwards, Caponetti et al. 2001). The concept is based on production of porous particles, by means of specific manufacturing processes and formulations, having a low density and thereby an aerodynamic diameter, dₐ, which is considerably smaller than the geometric diameter, d_{g}, according to the equation: (Hinds 1982)
dₐ=√\vec{ρ}d_{g}

It is the aerodynamic diameter of a particle that predicts the behavior of a particle in air flow streams. The smaller the aerodynamic diameter is, the larger is the probability of deposition far down into the lungs at inhalation e.g., in the alveoli. Due to larger geometric diameter porous particles have considerably better flowing properties than more compact particles having the same aerodynamic diameter. Besides the fact that they are more readily handled and disaggregate they have a longer residence time in the lung than small particles (Edwards, Hanes et al. 1997). The improved properties also lead to other technical advantages which may be of importance not least for fulfilling authority requirements, for example such particles will become easier to dose and mix. The positive technical features also facilitate to fulfil the inevitable and sometimes difficult to achieve requirements of the inhalation drugs with regard to amount and dose content uniformity (DCU).

Particles having a low density can be manufactured using substances approved by Food and Drug Administration (FDA), only (Vanbever, Mintzes et al. 1999). In order to obtain porous particles polymers are commonly used in the formulation (Hanes, Edwards et al. 1999; Edwards, Langer et al. 1999; Edwards, Caponetti et al. 1999; Edwards, Caponetti et al. 2001).

Other ways of manufacturing porous particles is to dry a mixture consisting of an emulsion of a “blowing agent”, e.g., fluorinated hydrocarbons, and a solution containing an active compound, (Tarara, Weers et al. 2000). In certain cases this concept requires two drying steps, (Weers, Tarara et al. 2001). Particles having a low density may also be manufactured using specific precipitation techniques. (Etter 2000)

### SUMMARY OF THE PRESENT INVENTION

The present invention utilizes the fact that certain agents in solution even at low concentrations (such as about or <1% dry matter) are able to associate into large three dimensional structures forming homogenous and thermodynamically stable viscous solutions. If the volatile agents are removed in a suitable manner the three dimensional structure formed by the agent is preserved, completely or partly, and particles having a low density are formed. For example, the removal of the volatile agents can take place by using spray or freeze drying.

Example of systems forming suitable solutions is phospholipids, organic solvent being in particular hydrophobic and volatile, and water in certain composition. The three dimensional structure consists in this case of longitudinally extending rodlike micelles and/or network of them.

In particular the present invention is characterized by an inhalable, porous free flowing particles suitable for use in therapeutic contexts, optionally containing one or more therapeutically active compound(-s) or substance(-s), whereby the particle consists of agents, which in diluted solutions self-associate to large three dimensional structures whereby after removal of volatile matter, such as by freeze drying, spray drying or other suitable evaporation method, they have a density of <0.5 g/cm³, preferably 0.001 to 0.5 g/cm³. A diluted solution herein means a solution comprising <10% (w/w) dry matter.

In a preferred embodiment the inhalable particle has a geometrical diameter, dg, of at least 30 μm.

In another preferred embodiment the inhalable particle has a geometrical diameter of 40 to 50 μm.

In a further preferred embodiment the inhalable particle has an aerodynamic diameter, (d,) of 0.5 to 5 μm whereby the aerodynamic diameter (dₐ) is preferably 1 to 5 μm.

In another preferred embodiment of the invention the particle comprises one or more therapeutically active compounds or substances being molecularly bound to the three dimensional network.

In a further preferred embodiment of the invention the particle comprises one or more therapeutically active compounds or substances adhered to the three dimensional network.

The invention disclosed herein advances the concept of porous particles further forward. It makes it possible to manufacture porous particles In a one-step process starting from a homogenous and equilibrated solution. The particles may have lower density than those previously described (density <0.01 g/cm³) and It Is possible to manufacture these consisting of constituent bodily lipids only. The simple manufacturing process may be used to facilitate aseptic manufacture, as well. The particles can be used as they are, or as carriers of active substance. In the case of phospholipids the particles are relatively chemically Inert. For example, they do not have any reducing properties as e.g., lactose has. This, together with the good particle properties they possess, makes them suitable as a mere dilution agent In the type of ordered mixtures of sensitive systems, such as certain proteins and peptides. When used as a carrier the active compound or substance can be dissolved in the solution from which the particle is manufactured, or be adhered thereto afterwards.

The present invention makes a new mixing concept possible. In ordinary so called ordered mixtures the large carrier particles serves mainly to make the powder manageable. In this new concept the carrier particles may serve as both flow enabler and as drug vehicle.

One decisive step when using ordered mixtures is the step when the small inhalable particles disaggregate from the noninhalable larger carrier particles. This critical step determines principally the amount of drug that gets Into the lungs. Systems with porous carrier particle however can be designed so that the need for this crucial step is eliminated or even gets unwanted. The low density of the carrier particle makes It possible to add substantial weight in form of for Instance drug particles without exceeding inhalable aerodynamic diameter. To a porous particles having the geometric diameter of 40 μm (density of 0.01 g/cm³) it is possible to add about 50% of weight without that the formed aggregate loose inhalable properties (aerodynamic diameter>5 μm).

For example, to a system of porous carrier particles with a geometric diameter of 40 μm (density of 0.01 g/cm³) It is possible to add around 5 drug particles (4 μm, density 1 g/cm³) per carrier particle (50%, w/w) and still have a aerodynamic diameter≈5 μm.

Alternatively, the carrier particle may serve as a vehicle for large noninhalable particles. Adding the same weight (50% w/w) in form of 7 μm particles (approx. 1 per carrier) to the 40 μm (density of 0.01 g/cm³) carrier particle results in same aerodynamic diameter (≈5 μm). It is of course also possible to add small low density (porous) particles. Particles may be added to the finished carrier particles or in one or more step(-s) in the manufacturing process, e.g., present in the start solution.

Typically drug particles will have a density of at least 0.5 g/cm3, more preferably at least 0.75 g/cm³, even more preferably 1.00 g/cm3, or more.

Start solution that Include an oil phase, surface active compound and water, e.g. lipid+water+oil, may be an advantage for pharmaceuticals that contains both a oil soluble and a water soluble component e.g. Symbicort® Turbuhaler® (Formoterol, water soluble, Budesonide, oil soluble).

It may be considered that the solution is also particularly well suited for the relatively frequent types of substances present having complicated dissolution properties. The solution can also have its advantages for pharmaceuticals which function In lipid matrixes, such as e.g., membrane proteins.

Large aggregates, in solution, built by small building blocks that are not covalently bound to each other have, unlike polymers, also the good property of being rebuild rapidly after having been subject to large shearing forces In e.g., pumps. In the case of solutions containing longitudinally extending aggregates it Is well known that this dampens the turbulence (Morgan and McCormick 1990) which in turn results in a reduced mechanical degradation of large molecules, as e.g., enzymes. By varying the dry matter content and other process parameters it is possible to control the resulting final density of the particles. This may be an advantage for applications, present and future, where an optimal density of particles is of importance.

In the case of lipids the solution is so called “reversed”, i.e., the hydrophobic (oil) phase is continuous. In this phase the lipids projects its hydrophobic part out into the continuous phase (oil) and its water-soluble part is directed towards a core of water. The solubility of lipids in oil is so low that this in many systems result in hydrophobic particles after the removal of the solvent. The hydrophobic surface reduces interactions with water. This may reduce or even eliminate problems with for instance particle growth and capillary condensation at high relative humidity. Capillary condensation complicates disaggregating in both ordered mixtures and spheronized systems.

In the case when the water phase is the continuous phase, the system is defined as “straight” In this description.

Powders of phospholipid particles formed according to the present invention turn out to have low interparticle forces resulting in good flow and disaggregating properties. This makes it possible to obtain large part of inhalable particles in DPI inhalators that merely accomplish low shear forces.

Relatively simple molecules, such as lipids, are from a general point of view, simpler to analyse and to follow their degradation of than in the case of polymers. Polymers are, furthermore, often very hard to manufacture within close specification ranges. This, together with the degradation (mechanical as well as chemical) during the manufacturing processes of pharmaceuticals often leads to a distribution in molecular size that can be hard to analyse. The advantages small molecules have over polymers is of vital importance when to fulfil the high and increasing authority demands with regard to purity, control of degradation and metabolism of pharmaceutical ingredients.

Although the present invention is particularly suitable for the pulmonary administration of bioactive agents it may also be used for the localized or systemic administration of compounds to any location of the body. Accordingly, it should be emphasized that, in preferred embodiments, the formulations may be administered using a number of different routes including, but not limited to, the gastrointestinal tract, the respiratory tract, topically, intramuscularly, intraperitoneally, nasally, vaginally, rectally, aurally, orally or ocular. More generally, the present invention may be used to deliver agents topically or by administration to a non-pulmonary body cavity. In preferred embodiments the body cavity is selected from the group consisting of the peritoneum, sinus cavity, rectum, urethra, gastrointestinal tract, nasal cavity, vagina, auditory meatus, oral cavity, buccal pouch and pleura.

The term “pulmonary” as used herein refers to any part, tissue or organ that is directly or indirectly involved with gas exchange, i.e., O₂/CO₂ exchange, within a patient. “Pulmonary” contemplates both the upper and lower airway passages and includes, for example, the mouth, nose, pharynx, oropharynx, laryngopharynx, larynx, trachea, carina, bronchi, bronchioles and alveoli. Thus, the phrase “pulmonary administration” refers to administering the formulations described herein to any part, tissue or organ that is directly or indirectly involved with gas exchange within a patient.

The particles are well suited for use in inhalation therapy. While the present invention may be embodied in many different forms, disclosed herein are specific illustrative embodiments thereof that exemplify the principles of the invention. It should be emphasized that the present invention is not limited to the specific embodiments as illustrated. The present invention may be pulmonary administrated by, but not limited to, dose inhalers, dry powder inhalers, atomizers, nebulizers or liquid dose instillation (LDI) techniques, or any other technique, to provide for effective drug delivery.

For example, the particles of the invention as such can be used to deliver surfactants to the lung of a patient. This is particularly useful in medical indications which require supplementing or replacing endogenous lung surfactants including, but not limited to, in the case of IRDS (Infant Respiratory Distress Syndrome) and ARDS (Adult Respiratory Distress Syndrome).

Formation of Particles

All systems that forms large three dimensional structures by non-covalent binding of small building blocks together in solutions is included in the present invention. It can be self assembled structures but may also be induced for instance by flow. Thus the present three dimensional structure is not based upon polymerized chains forming an extended non-covalent structure under polymerization.

Using the geometrical model proposed by Tartar (Tartar 1955) and further developed by others (Tanford 1973; Israelachvili, Mitchell et al. 1976; Mitchell, Tiddy et al. 1983; Israelachvili 1992) means that all building blocks that have or may form suitable packing parameter (or shape factor) Nₛ for creating such large three dimensional structures is included in the present invention.
Nₛ=v/(aₒI_{c})
v=hydrocarbon chain volume
aₒ=Optimal surface area
I_{c}=Critical hydrocarbon chain length

Specific examples of suitable structures of three dimensional network include, but are not limited to, long rodlike micellar systems. In the case of system were water is the continuous phase, building blocks having packing parameter Nₛ around 0.5, in particular 0.3 to 0.8, or more in particular 0.4 to 0.6, forms cylindrical (rodlike) micelles or micellar network. Similar cylindrical micelles or network may form when Nₛ is >1, preferably less than 0.75, more preferably less than 0.5. In this case when oil is the continuous phase the system is usually called reversed. When a reversed system is used a minor amount of water is generally used, whereby this amount is 1-30 moles per mole of structure forming material, preferably 1-20 moles per mole, or more preferably 1-10 moles per mole.

All solvents and all combinations thereof are included. They can be partially purified or fractionated to comprise pure fractions or mixtures. Examples of suitable solvents include, but not limited to, water, carbon dioxide, alkanes, alkenes, alkynes, carboxylic acids, benzenes, amines, nitro compounds, isocyanates, carbamates, thiols, sulphides, esters, phosphates, phosphines, phenols, phenyl ethers, aldehydes, ketones, carbohydrates, polycyclic aromatic hydrocarbons, heterocyclic compounds, halogenated hydrocarbons, polysiloxanes and all derivates of them.

Specific examples of suitable solvents include, but not limited to, water, carbon dioxide, pentane, hexane, heptane, octane, nonane, decane, undecane, dodecane, tridecane, tetradecane, pentadecane, hexadecane, heptadecane, dimethylbutene, hexene, octadiene, tripropylamin, tributylamine, triisobutylamine, trioctylamine, dibutyl ether, dodecenylsuccinic anhydride, ethyl laurate, butyl laurate, ethyl myristate, isopropyl paimitate, isooctane, cyclopentane, cyclohexane, cydoheptane, cyclooctane, cyclodecane, methyl cyclohexane, butylcyclohexane, phenylcyclohexane, bicyclohexyl, triisopropylbenzene, octylbenzene, decaline, and pinane.

Whatever building block is selected it will be appreciated that it may be used in its natural form, or as one or more salts known in the art.

Most types of surface active substances are able to build proper structures for the present invention. The structure may form in a pure solvent or it may be induced by for instance, but not limited to, high electrolyte concentration, a special counter ion, mixing different solvents, mixing surface active substances, mixing surface active substances with opposite charge or charge distribution, mixing with other substances that affect the packing and any combination of them.

Particle properties can of course be optimized by choosing ingredients e.g. building block(-s) with suitable properties. Example of this include, but not limited to, optimizing the particle melting properties by using lipids with suitable hydrocarbon chain length and degree of saturation.

Examples of suitable systems include, but not limited to, surfactant+organic solvent+Water, zwitterionic surfactant+anionic surfactant+water, cationic surfactant+salicylate+water, cationic surfactant+anionic surfactant water, zwitterionic surfactant+long alcohol+water. For more examples see (Hoffmann and Ebert 1988; Scartazzini and Luisi 1988; Harwigsson 1995) and references therein.

Preferably, the building block will comprise a phospholipid or other surfactant approved for pharmaceutical, especially pulmonary, use. Below are examples of suitable building blocks.

Lipids, both polar and non-polar, from both natural and synthetic sources are particularly compatible with the present invention and may be used in varying concentration to form suitable structures. They can be combined and mixed without any limitation. Examples of suitable lipids include but are not limited to those described in (Gunstone, Harwood et al. 1986) and references therein. They can be partially purified or fractionated to comprise pure fractions or mixtures like for instance lecithin. Constituent bodily lecithin can be used as well as egg lecithin and soya lecithin, whereby, however, constituent bodily lecithin and egg lecithin are preferred. Phospholipids are available from a variety of natural sources and may be synthesized by methods known in the art; see, for example, (Tsai 1988; Dennis 1993). A1) kinds of modifications of the listed compounds are also included. Examples include but are not limited to PEGylation. Lipids with all kinds of hydrocarbon chain configuration and modification is included.

Examples include but are not limited to saturated, unsaturated, branched and PEGylated chains.

Nonpolar lipids include, among others, triacylglycerols usually called triglycerides, diglycerides, sterols.

Polar lipids include, among others, monoglycerides, galactosylglycerolipids, sphingolipids, phosphoglycerolipids.

The galactosylglycerolipids includes, among others, monogalactosyldiglycerides (MGDGs), dlgalactosyldiglycerides (DGDGs).

Sphingolipid is a generic name for lipids having a long-chain base sphingoid such as glycosphingolipids, sphingophospholipids (involving sphingophosphonolipids) and ceramides. The sphingolipids includes, among others, sphingomyelin(SM),N-stearyl sphingomyelin, N-palmityl sphingomyelin, N-oleyl sphingomyelin, cerebroside.

The phosphoglycerolipids includes, among others, phosphatidylcholines (PC), phosphatidylethanolamines (PE), phosphatidylglycerols, phophatidylserines, phosphatidylinositols, phosphatidic acid and combinations thereof.

Specific examples of phospholipids include but are not limited to phosphatidylcholines such as dipalmitoyl phosphatidylcholines (DPPC), dimyristoyl phosphatidylcholines (DMPC), 1-stearoyl-2-palmitoyl phosphatidyl choline (SPPC), distearoyl phosphatidylcholines (DSPC), dilauroyl phosphatidyl choline, dioleoyl phosphatidyl choline, dilinoleoyl phosphatidyl choline and 1-palmitoyl-2-oleoyl phosphatidyl choline, dipentadecanoylphosphatidylcholine.

Phosphatidyl ethanol amines such as dipalmitoyl phosphatidylethanolamines (DPPE), 1,2-dioleoyl-3-n-phosphatidyl ethanolamine (DOPE), 1,2-distearoyl-3-n-phosphatidyl ethanolamine (DSPE), ), 1-stearoyl-2-palmitoyl phosphatidyl ethanolamine (SPPE) and 1,2-diphytanoyl-3-n-phosphatidyl ethanolamine (DiPyPE).

Phosphatidylglycerols such as dipalmitoyl phosphatidyl glycerol (DPPG),

Phophatidylserines such as 1,2-dioleoyl phophatidyl serine (DOPS), 1,2-dipalmitoyl phosphatidyl serine (DPPS).

Phosphatidylinositols such as 1,2-distearoyl phosphatidyl inositol (DSPI),

Phosphatidinic acids, such as dimyristoyl phosphatidinic acid and dipalmitoyl phosphatidinic acid.

Other lipids which are contemplated include but are not limited to: lipids such as fatty acid salt such as sodium caproate, sodium caprylate sodium caprate, sodium laurate, sodium myristate, sodium myristolate, sodium palmitate, sodium palmiltoleate, sodium oleate 18, sodium ricinoleate, sodium linoleate, sodium linolenate, sodium stearate, sodium arachidonate, the corresponding acid form and other salts and derivates of them also included, bile salt such as sodium cholate, sodium taurocholate, sodium glycocholate, sodium deoxycholate, sodium taurodeoxycholate, sodium glycodeoxycholate, sodium ursodeoxycholate, sodium chenodeoxycholate, sodium taurochenodeoxycholate, sodium glyco cheno deoxycholate, sodium ursodeoxycholate, sodium cholylsarcosinate, sodium N-methyl taurocholate the corresponding acid form and other salts and derivates of them also included, lysolipids such as lysophosphatidylcholine, lysophosphatidylglycerol, lysophosphatidylethanolamine, lysophosphatidylinositol, lysophosphatidylserine, glycolipids such as ganglioside GM1 and GM2; sulfatides; lipids bearing polymers such as polyethyleneglycol, chitin, hyaluronic acid or polyvinylpyrrolidone; lipids bearing sulfonated mono-, di-, oligo- or polysaccharides; cholesterol, cholesterol sulfate and cholesterol hemisuccinate; tocopherol hemisuccinate, lipids with ether and ester-linked fatty acids, polymerized lipids, diacetyl phosphate, stearylamine, cardiolipin, phospholipids with short chain fatty acids of 6-8 carbons in length, synthetic phospholipids with asymmetric acyl chains (e.g., with one acyl chain of 6 carbons and another acyl chain of 12 carbons), 6-(5-cholesten-3β-yloxy)-1-thio-β-D-galactopyranoside, digalactosyldiglyceride, 6-(5-cholesten-3β-yloxy)hexyl-6-amino-6-deoxy-1-thio-β-D-galactopyranoside, 6-(5-cholesten-3β-yloxy)hexyl-6-amino-6-deoxy--1-thio-α-D-manno pyranoside, 12-(((7′-diethylaminocoumarin-3-yl)carbonyl)methylamino)-octadecanoic acid; N-[12-(((7′-diethylaminocoumarin-3-yl)carbonyl)methyl-amino) octadecanoyl]-2-aminopalmitic acid; cholesteryl)4′-trimethyl-ammonio)butanoate; N-succinyldioleoylphosphatidylethanolamine; 1,2-dioleoyl-n-glycerol; 1,2-dipalmitoyl-n-3-succinylglycerol; 1,3-dipalmitoyl-2-succinylglycerol; 1-hexadecyl-2-palmitoylglycerophosphoethanolamine and palmitoylhomocysteine, dicetylphosphatel, phosphatidyl methanol, phosphatidyl-β-lactate and oleic acid, also cationic lipids of the type of the 1,2-diacyl-3-trimethyl ammonium propanes (TAP) or the 1,2-diacyl-3-dimethyl ammonium propanes (DAP) and/or combinations thereof.

Besides those surfactants enumerated above, it will further be appreciated that a wide range of surfactants may optionally be used in conjunction with the present invention. Examples of suitable surfactants include but are not limited to those described in (Hollis 1995) and references therein.

Examples of suitable surfactants include, but not limited to, surfactants without net charge like non-Ionic and zwitterionic, charged surfactants such as anionic and cationic surfactants and amphoteric surfactants. The hydrophobic groups in these surfactants may include, but not limited to, aliphatic, aromatic or alicyclic hydrocarbons, fluorocarbons or polysiloxanes.

Examples of suitable non-ionic surfactants include, but not limited to, fatty acid alkanolamides, alkoxylated alcohols preferably ethoxylated alcohols, tertiary amine oxides, ethoxylated castor oil, ethoxylated fatty acid alkanolamides, ethoxylated N-alkylamines, ethoxylated and alkoxylated alkyl phenols, ethoxylated fatty acid esters, lanolin based derivatives, betaine derivatives, pentaerythritol derivatives, sterol derivates such as derivats of phytosterol and cholesterol, sorbitan derivatives, sucrose esters and polyglycosides derivatives, other suger surfactants, other surfactants containing one or several polyoxyethylene groups.

More specific examples of suitable nonionic surfactant include, but are not limited to, nonionic surfactants with polyoxyethylene headgroup such as C₈₋₂₄ alcohol+ethylene oxide, fatty acid (e.g. from rapeseed, olive, corn, sunflower, cotton seed oil) monoethanolamide+ethylene oxide(FAAE), sorbitan trioleate, sorbitan mono-oleate, sorbitan monolaurate, polyoxyethylene sorbitan monolaurate

Sucrose fatty acid esters include monoesters, diesters and triesters of sucrose, or mixtures or blends thereof. Specific examples include, but not limited to, sucrose monolaurate, sucrose monomyrstate, sucrose monopalmitate, sucrose monostearate, sucrose distearate, sucrose tristearate, sucrose trimyristate, and sucrose tripaimitate.

More specific examples of suitable zwitterionic surfactant include, but not limited to, C₈₋₂₄ betaines, C₈₋₂₄ sulphobetaines,

Examples of suitable anionic surfactants include, but not limited to, alkylsulphonates, alkyl aryl sulphonates, alkyl ether sulphates, alkyl sulphates, carboxylic and polycarboxylic derivatives, diaryl sulphonate derivatives, acylisothionates, naphthalene sulphonates, olefin sulphonates, mono- and dialkylphosphates, sarcosinates, fatty acid esters of taurates, taurinates, dialkylsulfosuccinates, N-alkylsuccinamates, sulphates and sulphonates of ethoxylated alkyl phenol, sulphates and sulphonates of oils and fatty esters, alpha-olefin sulphonates.

More specific examples of suitable anionic surfactants include, but are not limited to, C₈₋₂₄ alkylbenzensulphonate, sodium lauryl sulphate (SDS), disodium monodocosyl sulfosuccinate, disodium monoundecylenethanolamidesulfosuccinate.

Examples of suitable cationic surfactants include, but not limited to, ethoxylated fatty amines, tertiary amine oxides, higher alkyl amine salts, lecithin derivatives, surfactants based on proteins, surface active quaternary ammonium compounds, polyamines, primary fatty amines, secondary fatty amines, tertiary fatty amines.

More specific examples of suitable cationic surfactants include, but are not limited to, C₈₋₂₄ alkyltrimethylammonium salts and C₈₋₂₄ alkylpyridinium salts.

More specific examples of suitable amphoteric surfactants include, but not limited to, C_{8 24} sarcosinates, C₈₋₂₄ imidazolines.

Example of commercial products according to different trademarks suitable for use in the present invention: TRITON X, TERGITOL, BRIJ, TWEEN, SPAN, POLYSORBATE, PLURONIC, SOLUTOL, SURFACTIN, TETRONICS.

Production

Among other methods, particles compatible with the instant invention may be formed by, techniques including, but not limited to, spray drying, vacuum drying, freeze drying, extrusion, or other suitable techniques and combinations thereof.

Other components or blends of other particles can be added to the particles of the present inventions by, but not limited to, blending, mixing, coating technique such as employed using a fluidized bed, spray drying of two solutions using a double nozzle technique.

Drugs

As used herein, an active agent includes an agent, drug, compound, and composition of matter or mixture thereof which provides some diagnostic, prophylactic, or pharmacologic, often beneficial, effect. Accordingly, an active agent optionally includes a detectable label (e.g., a radioactive label) that is useful for identifying the locations of the released agent in vivo; active agents also include therapeutic agents which are useful for treating a disease or condition. This includes nutrients, nutritional factors, drugs, vaccines, vitamins, and other beneficial agents. As used herein, the terms further include any physiologically or pharmacologically active substance that produces a localized or systemic effect in a patient. In certain embodiments, the preferred physiologically active agents are protein or peptide agents. Such protein or peptide agents typically can be further divided into categories, based upon the activity of the agent or the type of disease or condition that is being treated.

The physiologically active agent which can be used in the present invention includes but is not limited to categories of antibiotics, antibodies, anepileptics, antiallergics, bronchodilators, bronchoconstrictors, pulmonary lung surfactants, leukotriene inhibitors or antagonists, anticholinergics, anaesthetics, antituberculars, imaging agents, haematopoietic agents, anti-infective agents, antidementia agents, antiviral agents, antitumoral agents, antipyretics, analgesics, anti-inflammatory agents, antiulcer agents, antiallergic agents, antidepressants, psychotropic agents, cardiotonics, antiarrythmic agents, vasodilators, antihypertensive agents such as hypotensive diuretics, antidiabetic agents, anticoagulants, cholesterol lowering agents, cytostatics, fungi-statics, free-radical scavengers, vitamins, hormones, immunostimulants, immunosuppressants, mucolytics, heparin, analgesics, soporifics and the like, anticholinergics, cyclooxygenase, mast cell, lipoxygenase and proteolytic enzyme inhibitors, arachidonic acid, leukotriene, thromboxane, sodium/potassium channel, neurokinin, tachykinin, bradykinin, muscarine, histamine, phosphodiesterase and selectin antagonists, potassium channel blockers, anti-infective agents, antibiotics, therapeutic agents for osteoporosis, hormones, vaccines, psychic energizers, tranquilizers, anticonvulsants, muscle relaxants, antiparkinson agents, muscle contractants, antimicrobials, antimalarials, hormonal agents including contraceptives, sympathomimetics, drugs capable of eliciting physiological effects, diuretics, lipid regulating agents, antiandrogenic agents, antiparasitics, neoplastics, antineoplastics, hypoglycemics, nutritional agents and supplements, growth supplements, fats, antienteritis agents, electrolytes, cardiovascular agents, enzymes, steroids, genetic material, viral vectors, viruses, antisense agents, proteins, peptides peptides and combinations thereof and, may be inorganic and organic compounds, including, without limitation, drugs which act on the peripheral nerves, adrenergic receptors, cholinergic receptors, the skeletal muscles, the cardiovascular system, smooth muscles, the blood circulatory system, synaptic sites, neuroeffector junctional sites, endocrine and hormone systems, the immunological system, the reproductive system, the skeletal system, autacold systems, the alimentary and excretory systems, the histamine system and the central nervous system.

While specific examples of active agents (e.g., peptide and non-peptide agents) for use in accordance with this invention are mentioned below, this does not mean that other peptide or non-peptide agents are excluded. These active agents may be naturally occurring, recombinant or chemically synthesized substances or in some form of prodrug or similar.

The preferred physiologically active peptide agents include peptide hormones, cytokines, growth factors, factors acting on the cardiovascular system, factors acting on the central and peripheral nervous systems, factors acting on humoral electrolytes and hemal organic substances, factors acting on bone and skeleton, factors acting on the gastrointestinal system, factors acting on the immune system, factors acting on the respiratory system, factors acting on the genital organs, and enzymes.

Exemplary hormones include insulin, growth hormone, human growth hormone (hGH), growth hormone releasing hormone (GHRH), alpha-1 proteinase inhibitor, elcatonin, parathyroid hormone, luteinizing hormone-releasing hormone (LH-RH), adrenocorticotropic hormone (ACTH), amylin, oxytocin, luteinizing hormone, (D-Tryp6)-LHRH, nafarelin acetate, leuprolide acetate, follicle stimulating hormone, glucagon, prostaglandins, PGE1, PGE2 and other factors acting on the genital organs and their derivatives, analogs and congeners. As analogs of said LH-RH, such known substances as those described in U.S. Pat. Nos. 4,008,209, 4,086,219, 4,124,577, 4,317,815 and 5,110,904 can be mentioned.

Exemplary antibiotics include tetracycline, aminoglycosides, penicillins, cephalosporins, sulfonamide drugs, chloramphenicol sodium succinate, erythromycin, vancomycin, lincomycin, clindamycin, nystatin, amphotericin B, amantidine, idoxuridine, p-amino salicyclic acid, isoniazid, rifampin, antinomycin D, mithramycin, daunomycin, adriamycin, bleomycin, vinblastine, vincristine, procarbazine, imidazole carboxamide, macrolides, quinolines, streptomycin, tetracyclines, pentamidine, amoxilline, azithomycine, clarithromycine, doxycycline, erythromycine, fluconazole, levofloxacine, minocycline, moxifloxacine, ofloxacine, pivampicilline.

Exemplary hematopoietic or thrombopoietic factors include, among others, erythropoietin, granulocyte colony stimulating factor (G-CSF), granulocyte-macrophage stimulating factor (GM-CSF) and macrophage colony stimulating factor (M-CSF), leukocyte proliferation factor preparation (Leucoprol, Morinaga Milk), thrombopoietin, platelet proliferation stimulating factor, megakaryocyte proliferation (stimulating) factor, and factor VIII.

Exemplary antidementia agents include selegelene.

Exemplary antiviral agents include amantidine and protease inhibitors.

Exemplary antitumoral agents include doxorubicin, Daunorubicin, taxol, and methotrexate.

Exemplary antipyretics and analgesics include aspirin, Motrin, Ibuprofin, naprosyn, indocin, and acetaminophen.

Exemplary antiinflammatory agents include NSAIDS, aspirin, steroids, dexamethasone, hydrocortisone, prednisolone, Diclofenac Na, fluticasone propionate, beclomethasone dipropionate, flunisolide, budesonide, tripedane, cortisone, prednisone, prednisilone, dexamethasone, betamethasone, triamcinolone acetonide, naproxen sodium, flurbiprofen, diclofenac sodium, diclofenac potassium, misoprostil, valdecoxib, celecoxib, sulindac, oxaprozin, salsalate, diflunisal, piroxicam, indomethacine, etodolac, meloxicam, ibuprofen, ketoprofen, nabumetone, tolmetin sodium, choline magnesium trisalicylate, rofecoxib,

Exemplary antiulcer agents include famotidine, cimetidine, nizatidine, ranitidine and sucralfate.

Exemplary antiallergic agents include antihistamines, methapyrliene, diphenydramine, loratadine, and chlorpheniramine.

Exemplary antidepressants and psychotropic agents include lithium, amitryptaline, venlafaxine, pheneizine, tranylcypromine, mirtazepine, nefazodone, triazolopyridine, bupropion, tricyclic antidepressants such as amitriptyline, desipramine, nortriptyline Selective Serotonin Reuptake Inhibitors (SSRI) such as citalopram, fluvoxamine, paroxetine, fluoxetine, sertraline. escitalopram.

Exemplary cardiotonics include digoxin.

Exemplary antiarrythmic agents include metoprolol and procainamide.

Exemplary vasodilators include nitroglycerin, nifedipine, and isosorbide dinitrate.

Examplary mast cell inhibitors are cromoglycic acid, nedocromil etc. and lipoxygenase inhibitors such as zileuton,

Examplary leukotriene antagonists are iralukast, zafirlukast and pranlukast, sodium channel antagonists are amiloride, potassium channel antagonists are bimakalim, arachidonic acid antagonists are 2-benzoxazolamine, histamine receptor antagonists are epinastine, cetrizine, mizolastine and mequitamium,

Examplary antimigraine agents are ergot alkaloids, methysergide, ergotamine, serotonin, sumatriptan, zolmitriptan, cyclandelate, almotriptan etc.

Examplary analgesics are fentanyl, morphine, buprenorphine, opium, heroin, nalbuphine, pentazocine, oxycodone, tramadol, pethidine, tilidine, methadone, nefopam, dextropropoxyphene, piritramide, codeine, dihydromorphine, ergotamine etc.

Examplary mucolytics are RNase, acetylcysteine, ambroxol, apafant, bromhexine, surfactant etc.

Examplary antiemetics are bromopride, domperidone, metoclopramide, triethylperazine, trifluoropromazine, meclozine, chlorphenoxamine, dimenhydrinate etc.

Exemplary diuretics include hydrochlorothiazide, amiloride and furosemide.

Exemplary antihypertensive agents include captopril, nifedipine, and atenolol. Exemplary antidiabetic agents include glucozide, chloropropamide, metformin, insulin, pioglitizone, rosigiltazone, glimepiride, sulfonlyurea, metformin, glyburide, miglitol, glipizide, repaglinide, acarbose, troglitazone, nateglinide.

Exemplary anticoagulants include warfarin, heparin, and Hirudin.

Exemplary anticholinergics and spasmolytics include atropine, scopolamine, N-butylscopolamine, trospium chloride, ipratropium bromide, oxitropium bromide, thiotropium bromide, drofenine, oxybutinine, moxaverine, glycopyrrolate etc.

Exemplary lungsurfactants include Surfaxin, Exosurf, Survanta

Exemplary antitussives include noscapine.

Exemplary antihistamines include fexofenadine, allegra, decongestant, desioratadine, loratidine, tecastemizole

Exemplary cholesterol lowering agents include lovastatin, cholestyamine, rosuvastatin, clofibrate. colestipol, fluvastatine, atorvastatin, niacin, pravastatin sodium, cholestyramine resin, fenofibrate, colesevelam hydrochloride, simvastatin, ezetimibe

Exemplary therapeutic agents for treating osteoporosis and other factors acting on bone and skeleton include calcium, alendronate, bone GLa peptide, parathyroid hormone and its active fragments (osteostatin), histone H4-related bone formation and proliferation peptide (OGP) and their muteins, derivatives and analogs thereof.

Exemplary enzymes and enzyme cofactors include: pancrease, L-asparaginase, hyaluronidase, chymotrypsin, trypsin, tPA, streptokinase, urokinase, pancreatin, collagenase, trypsinogen, chymotrypsinogen, plasminogen, streptokinase, adenyl cyclase, and superoxide dismutase (SOD).

Exemplary vaccines include Hepatitis B, MMR (measles, mumps, and rubella), and Polio vaccines.

Exemplary Immunological adjuvants include: Freunds adjuvant, muramyl dipeptides, concanavalin A, BCG, and levamisole.

Exemplary cytokines include lymphokines, monokines, hematopoletic factors and so on.

Lymphokines and cytokines useful in the practice of the invention include interferons (e.g., Interferon-alpha, -beta and -gamma), interleukins (e.g. interleukin 2 through 11) and so on. Monokines useful in the practice of the invention include interleukin-1, tumor necrosis factors (e.g. TNF-alpha and -beta), malignant leukocyte inhibitory factor (LIF) and so on.

Exemplary growth factors include nerve growth factors (NGF, NGF-2/NT-3), epidermal growth factor (EGF), fibroblast growth factor (FGF), insulin-like growth factor (IGF), transforming growth factor (TGF), platelet-derived cell growth factor (PDGF), hepatocyte growth factor (HGF) and so on.

Exemplary factors acting on the cardiovascular system include factors which control blood pressure, arteriosclerosis, etc., such as endothelins, endothelin inhibitors, endothelin antagonists (such as described in EP 436189, 457195, 496452 and 528312), endothelin producing enzyme inhibitors vasopressin, renin, angiotensin I, angiotensin II, angiotensin III, angiotensin I inhibitor, angiotensin II receptor antagonist, atrial naturiuretic peptide (ANP), antiarrythmic peptide and others.

Exemplary factors acting on the central and peripheral nervous systems include oploid peptides (e.g. enkephalins, endorphins), neurotropic factor (NTF), calcitonin gene-related peptide (CGRP), thyroid hormone releasing hormone (TRH), salts and derivatives of TRH (U.S. Pat. No. 3,959,247), neurotensin and so on.

Exemplary factors acting on the gastrointestinal system include secretin, omeprazole and other gastric acid secretion influencing drugs, and gastrin.

Exemplary factors acting on humoral electrolytes and hemal organic substances include factors which control hem aglutination, plasma cholesterol level or metal ion concentrations, such as calcitonin, apoprotein E and hirudin. Laminin and intercellular adhesion molecule 1 (ICAM 1) represent exemplary cell adhesion factors.

Exemplary factors acting on the kidney and urinary tract include substances which regulate the function of the kidney, such as brain-derived naturiuretic peptide (BNP), urotensin, DDAVP and so on.

Exemplary factors which act on the sense organs include factors which control the sensitivity of the various organs, such as substance P.

Exemplary factors acting on the immune system include factors which control inflammation and malignant neoplasms and factors which attack infective microorganisms, such as chemotactic peptides and bradykinins.

Exemplary factors acting on the respiratory system include factors associated with asthmatic responses.

Also included are naturally occurring, chemically synthesized or recombinant peptides or proteins which may act as antigens, such as cedar pollen and ragweed pollen. These factors are administered, either independently, coupled to haptens, or together with an adjuvant, in the formulations according to the invention.

In the same way naturally occurring antibodies, chemically synthesized or recombinant antibodies can be administered for passive immunization purposes.

Active agents may further comprise nucleic acids, present as bare nucleic acid molecules, viral vectors, associated viral particles, nucleic acids associated or incorporated within lipids or a lipid-containing material, plasmid DNA or RNA or other nucleic acid construction of a type suitable for transfection or transformation of cells, particularly cells of the alveolar regions of the lungs.

The formulation may also include but is not limited to a content of a beta-mimetic selected from the group consisting of ephedrine, metaproterenol, albuterol, salbutamol, formoterol, salmeterol, fenoterol, clenbuterol, terbutaline, bambuterol, broxaterol, epinephrine, isoprenaline, orciprenaline, hexoprenaline, tulobuterol, reproterol, bamethan, rimiterol, reproterol, adrenaline, pirbuterol, bitolterol, procaterol, picumeterol, 8-hydroxy-5-(1-hydroxy-2-((2-(4-methoxyphenyl)-1-methylethyl)amino)ethyl)-2(1H)-quinoline, mabuterol, trianicinolone, acetonide, mometasone, and pharmacologically acceptable esters, salts and solvates of these compounds, anticholineigic bronchodilators, for example ipratropium bromide

The formulation may include but is not limited to a content of a corticoid selected from the group consisting of beclomethasone, betamethasone, ciclomethasone, dexamethasone, triamcinolone, budesonide, butixocort, ciclesonide, fluticasone, flunisolide, icomethasone, mometasone, tixocortol, loteprednol, tipredane, dexamethasone, fluocinolone, rofleponide and pharmaceutically acceptable salts thereof. For example salbutamol and terbutaline may be used as the sulphate; fenoterol as the hydrobromide; salmeterol as the xinafoate; formoterol as the fumarate dihydrate; clenbuterol as the hydrochloride; fluticasone as the propionate; and broxaterol as the monohydrochloride.

For the topical application of active compounds in the area of the bronchi and bronchioles, particle sizes of about 2-4 μm are advantageous, such as are customarily achieved with suspension formulations. Smaller particles which reach the alveolar area are partly exhaled (<0.5 μm) or reach the systemic circulation as a result of absorption. It follows from this that aerosol preparations for systemic application favourably should have particle sizes of about 0.5 μm-2 μm, where, for example, a monodisperse aerosol having a very high proportion of particles in the range of about 1 μm would be particularly advantageous. Depending on the desired deposition site, a smaller or larger MMAD and optionally a monodisperse distribution spectrum is therefore preferred. With respect to the aerodynamics: the greater the mass of the particles the larger their tendency to continue flying in a straight line. It results from this that in the case of a change in the flow direction impaction of particles occurs. It is known from deposition studies that even with an optimal inhalation manoeuvre only about 20% of the particles emitted from a metered aerosol reach the lungs and almost 80% impact in the oropharynx.

Other useful compound include but not limited by, erythropoietin (EPO), Factor VIII, Factor, ceredase, cerezyme, cyclosporine, elcatonin, heparin, low molecular weight heparin (LMWH), interleukin-2, luteinizing hormone releasing hormone (LHRH), leuprolide, somatostatin, somatostatin analogs including octreotide, vasopressin analog, follicle stimulating hormone (FSH), immunoglobulins, insulin-like growth factor, insulintropin, nerve growth factor, parathyroid hormone (PTH), thymosin alpha 1, IIbIIIIa inhibitor, alpha-1 antitrypsin, respiratory syncytial virus antibody, cystic fibrosis transmembrane regulator (CFTR) gene, deoxyribonuclease (Dnase), bactericidal/permeability increasing protein (BPI), anti-CMV antibody, interleukin-1 receptor, 13-cis retinoic acid, nicotine, nicotine bitartrate, codeine, caffeine, nicotine, gentamicin, ciprofloxacin, amikacin, tobramycin, metaproterenol sulfate, glucagon, LHRH, nafarelin, goserelin, leuprolide, interferon, rhu II-1 receptor, macrophage activation factors such as lymphokines and muramyl dipeptides, oploid peptides and neuropeptides such as enkaphalins, endophins, renin inhibitors, cholecystokinins, DNAse, growth hormones, leukotriene inhibitors and the like. In addition, bioactive agents that comprise an RNA or DNA sequence, particularly those useful for gene therapy, genetic vaccination, genetic tolerization or antisense applications, immunomodulators, anti-allergic drugs for example sodium cromoglycate and nedocromil sodium; expectorants; mucolytics; antihistamines; cyclooxygenase inhibitors; leukotriene synthesis inhibitors; leukotiene antagonists, PLA2 Inhibitors, PKC-inhibitors, PAF antagonists, aminophylline, montelukast, oxtriphyline, theophylline, cefaclor, cefadroxil, cefuroxime axetil, ciprofloxacin hydrochloride, pneumococcal conjugate vaccine, pneumococcal polysaccharide vaccine, prednisone, omeprazol, esomeprasol, sildenafil, vardenafil, tadalafin, and prophylactics of asthma; or pharmacologically acceptable esters and salts and/or solvates thereof.

For example, the particles of the invention as such can be used to deliver surfactants to the lung of a patient. This is particularly useful in medical indications which require supplementing or replacing endogenous lung surfactants including, but not limited to, in the case of IRDS (Infant Respiratory Distress Syndrome) and ARDS (Adult Respiratory Distress Syndrome).

The active compounds mentioned can optionally be used in the form of their isomers, enantiomers or racemates and, in the case of acids or bases, as such or in the form of their pharmaceutically acceptable salts. The optimum amount of active compound in the formulations according to the invention depends on the particular active compound. As a rule, however, aerosol formulations are preferred which contain at least approximately 0.0001 and at most approximately 5% by weight, in particular approximately 0.01 to 3% by weight, of active compound. However, in the present case the particle as such may serve as a pharmaceutical or therapeutical agent in the lung, and in that case the formulation is 100% active compound. There is reason to believe that in certain cases more than 5% by weight can be added to the particle of any other active ingredient, whereas the total amount may reach 20%, or even 30% or even up to 50% or more per weight.

Excipients

It will also be understood that other component(s) can be included in the present invention. Such component(s) may be added directly to the suspension medium or associated with, or incorporated in, the present invention. In any event, the excipient(-s) may be associated with, or incorporated in the present invention in any form. The present invention may comprise, incorporate, adsorb, absorb, be coated with or be partly formed by the excipient(s).

Such optional excipients include, but are not limited to: osmotic agents, stabilizers, chelating agents, colouring agents, taste masking agents, buffers, hygroscopic agents, antioxidants, viscosity modulators, salts, sugars or blend or other combination thereof.

For example excipients can be added to fine tune the present invention for maximum life and ease of administration.

In general, buffer substances or stabilizers such as citric acid, EDTA, vitamin E and the like substances of this type, if present, are used in amounts of not more than approximately 1% by weight, for example approximately 0.0001 to 1% by weight, based on the total formulation.

Compatible excipients may include, but are not limited to, carbohydrates including monosaccharides, disaccharides and polysaccharides. For example, monosaccharides such as dextrose (anhydrous and monohydrate), galactose, mannitol, D-mannose, sorbitol, sorbose and the like; disaccharides such as lactose, maltose, sucrose, trehalose, and the like; trisaccharides such as raffinose and the like; and other carbohydrates such as starches (hydroxyethylstarch), cyclodextrins, maltodextrins and hyaluronic acid. Amino adds are also suitable excipients. The inclusion of both inorganic (e.g. sodium chloride, calcium chloride, etc.), organic salts (e.g. sodium citrate, sodium ascorbate, magnesium gluconate, sodium gluconate, tromethamine hydrochloride, etc.) and buffers is also contemplated. The inclusion of salts and organic solids such as ammonium carbonate, ammonium acetate, ammonium chloride or camphor are also contemplated.

The present invention may also include a biocompatible, preferably biodegradable polymer, copolymer, or blend or other combination thereof.

X-Ray Contrast Agents:

The three dimensional network can be used as an X-ray contrast composition when combined with an X-ray contrast agent. Such an X-ray contrast agent can be used for imaging the bronchi and alveolar structures of the lung, e.g., for determining different types of emphysema.

Besides mere X-rays blocking agents even air bubbles and other particles can be used as contrast agents. Thus the present invention is ideal to provide a composition having particles with large air content.

The present network forming substances, including phospholipids, can be a carrier for such X-ray contrast agents to facilitate X-ray imaging of lungs.

Examples of suitable materials for use as contrast agents in MRI include but are not limited to the gadolinium chelates currently available, such as diethylene triamine pentacetic acid (DTPA) and gadopentotate dimeglumine, as well as iron, magnesium, manganese, copper and chromium as well as gamma-camera teknesium peikmetate.

Diagnostic agents also include but are not limited to imaging agents which include commercially available agents used in positron emission tomography (PET), computer assisted tomography (CAT), single photon emission computerized tomography, x-ray, fluoroscopy, and magnetic resonance imaging (MRI).

Microbubbles in the form of a gas emulsion can be used as ultrasound contrast enhancement agent. (Klein, Trevino et al. 1996)

The present invention low density particles consists mainly of air and is therefore suitable for the use as ultrasound contrast enhancement agents. By using suitable building blocks and manufacturing process it is possible to make particles that survive multiple passes through the entire circulatory system of a patient following intravenous injection. This makes it possible to administer small non-toxic doses in a peripheral vein and use it to enhance images of the entire body.

The good flow properties and the fat character of lipid particles fabricated according to the present invention makes them suitable as lubricants, like magnesium stearate, for instance when tabletting.

Although preferred embodiments of the present invention comprise powders and stabilized dispersions for use in pharmaceutical applications, it will be appreciated that the microstructures and disclosed dispersions may be used for a number of non pharmaceutical applications. That is, the present invention provides microstructures which have a broad range of applications where a powder is suspended and or aerosolized. In particular, the present invention is especially effective when low density particles is needed and where an active or bioactive ingredient must be dissolved, suspended or solubilized as fast as possible. By increasing the surface area of the porous microparticles or by incorporation with suitable excipients as described herein, will result in an improvement in dispersibility, and or suspension stability. In this regard, rapid dispersement applications include, but are not limited to: detergents, dishwasher detergents, food sweeteners, condiments, spices, mineral flotation detergents, thickening agents, foliar fertilizers, phytohormones, insect pheromones, insect repellents, pet repellents, pesticides, fungicides, disinfectants, perfumes, deodorants, etc.

The present invention offers benefits over prior art preparations for use in applications which require aerosolization or atomization. In such non pharmaceutical uses the preparations can be in the form of a liquid suspension (such as with a propellant) or as a dry powder. Preferred embodiments comprising the present invention as described herein include, but are not limited to, ink jet printing formulations, powder coating, spray paint, spray pesticides, inorganic pigments, dyes, inks, paints, explosives, pyrotechnic, adsorbents, absorbents, catalyst, nucleating agents, polymers, resins, insulators, fillers, etc.

### SHORT DESCRIPTION OF THE FIGURES

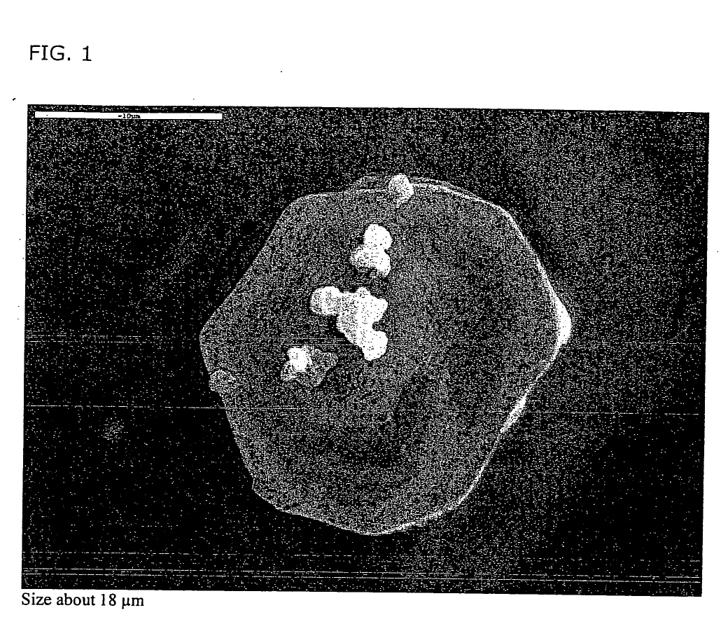

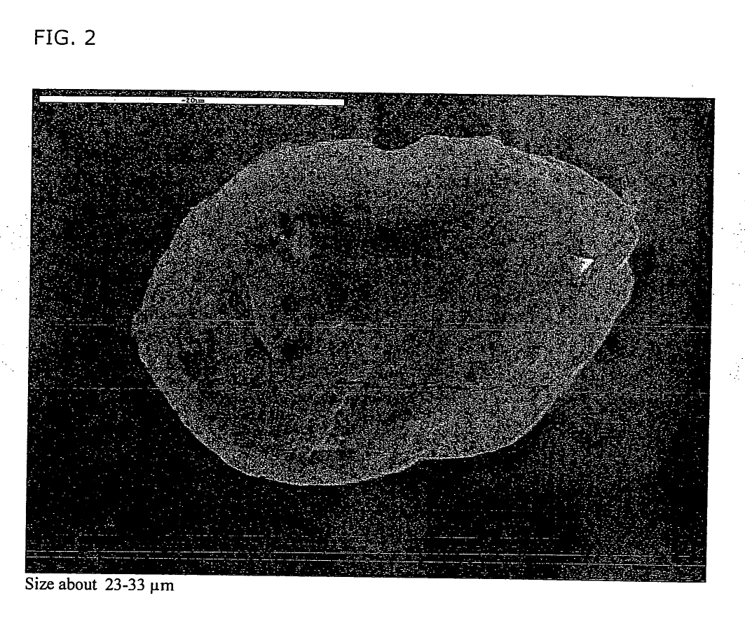

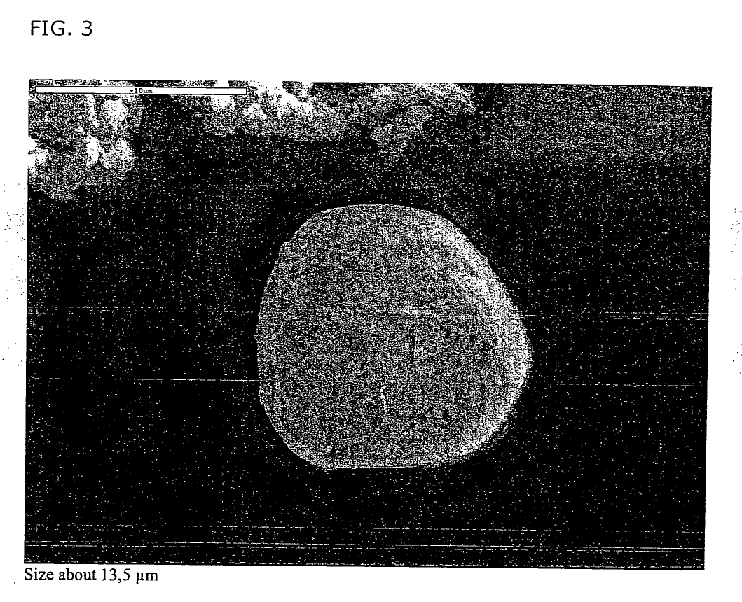

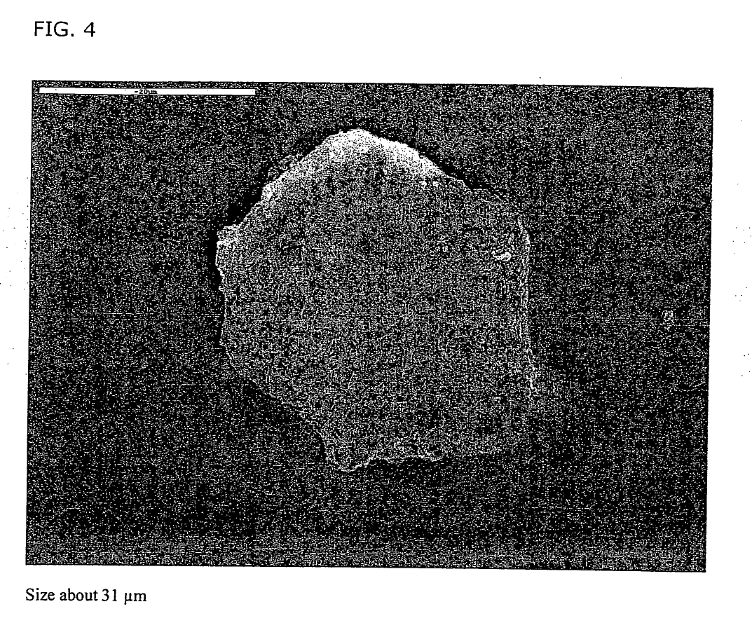

Particles of the present invention are shown in the attached sweep electron microscope images, which particles have been caught in an ANDERSEN cascade impacter (Mark II andersen 1 ACFM Non Viable Ambient Particle Sizing Sampler, graseby-Andersen, Smyma, Ga.) at step 4, FIG. 1-2, and at step 6, FIG. 3-4, respectively.

### DETAILED DESCRIPTION OF THE INVENTION

Impacters such as, for example, the 5-stage multistage liquid impinger (MSLI) or the 8-stage Andersen cascade impacter (ACI), which are described in chapter 601 of the United States Pharmacopoeia (USP) or in the Inhalants Monograph of the European Pharmacopoeia (Ph. Eur.), are suitable for testing the particles of the present invention. Particles according to the present invention have been tested in an ANDERSEN impacter.

The Andersen impacter is a standard instrument for the testing of inhalation products. The Andersen Impacter is an apparatus in which one determines the aerodynamic size of an aerosol. The principle is that one increases the speed of an air flow comprising particles in several steps. In each step the air flow is diverted which makes particles having a large aerodynamic diameter will not accompany the air flow but will impact onto a disc present on each step. The particles can then be collected from each disc and be analysed with respect to density, aerodynamic size and geometric diameter.

In the case of phospholipids, water and oil the amount of water is critical for the formation of large three dimensional network structures in a solution. The amount of water needed to form large structures has an upper and a lower limit depending on the system, i.e., type of oil and type of lipid and other components. The amount is in the range of 1 to 20 moles of water per mole of lipid.

A suitable amount of water for use in a particular formulation may be determined by standard methods such as cryo-TEM.

### EXAMPLE 1

A solution consisting of 7 g of DPPC, dipalmitoylphosphatidylcholine, (CAS nr: 63-89-8) and 3 g of DMPC, dimyristoylphosphatidylcholine (CAS nr: 18194-24-6) was mixed with 630 μl of water (Milli-Q) and 1000 ml of N-hexane (CAS 110-54-3). The total solution was mixed while stirred and was heated (50° C.) until a viscous solution was obtained.

The solution was dried in a spray drier of the mark MOBILE MINOR™ från GEA Niro A/S, having a mass flow of 4.9 kg/hr, a solution temperature of 50° C. and using nitrogen (N₂) as a drying gas. Ingoing temperature was thus 50° C., while outgoing temperature was 36° C.

The product obtained consisted of porous low density particles having a large three dimensional network having an aerodynamic diameter of 5 μm a geometric diameter of 50 μm and a density≈0.01 g/cm³.

In the accompanying photographs, SEM, particles are shown which have attached to step 4, 2 particles, (FIGS. 1-2), and step 6, 2 particles (FIGS. 3-4) in an ANDERSEN Impactor. The test conditions were thus such that on step 4 particles having a aerodynamic diameter of 1,4-2,3 μm and on step 6 particles having the aerodynamic diameter 0.5-0.8 μm are attached. The particles on the photographs have a geometrical diameter that is 10 times or more, larger than the aerodynamic diameter that normally get caught on theses steps. The particles are, as evident from the pictures, no perfect spheres, so a part of the explanation that they attach to these steps can be explained by the form factor, but the dominating reason why so large particles attach so far down in the impactor is that they have a considerably lower density. The theoretical relationship between the aerodynamic diameter (dₐ) and the geometrical diameter (d_{g}), as mentioned above, is given by the following relationship:
dₐ=√\vec{ρ}d_{g}

Andersen test conditions were: suction time 4 s; flow 60 l/min, and temperature 23° C., at 30% RH.

The solution which was dried contained about 1% dry matter. At a maintained volume after drying, this results in particles having a density of about 0.01 g/cm³. This means, that at equal aerodynamic diameters, these particles are about ten times larger than particles having the density 1 g/cm³. This in turn leads to the fact that it is possible to manufacture inhalable particles (dₐ=0.5-5 μm) which also will become free flowing (normally >40 μm).

### EXAMPLE 2

This example shows a manufacture essentially as in Example 1 but in a smaller scale and using a smaller spray drier.

A solution of 0.3505 g of DPPC, dipalmitoylphosphatidylcholine, (CAS nr: 63-89-8) was mixed with 0.1501 g of DMPC, dimyristoylphosphatidylcholine (CAS nr: 18194-24-6), and 32.5 μl of water (Milli-Q). 50 ml of N-hexane (CAS 110-54-3) were added. The solution was mixed while being stirred and heated (52° C.) until a viscous solution was obtained.

The solution was dried using a spray drier of the mark SDMicro™ of GEA Niro A/S, using a mass flow of 400 g/hr, a solution temperature of 52° C. and nitrogen (N2) as drying gas, whereby the ingoing temperature was 52° C., and outgoing temperature was 37° C. The product obtained consisted of porous low density particles having a large three dimensional network having an aerodynamic diameter of 5 μm a geometric diameter of 50 μm and a density≈0.01 g/cm³.

### EXAMPLE 3

A solution consisting of 2 g of DMPC (CAS nr:18194-24-6) and 1 g DPPC(CAS nr: 63-89-8) was mixed with 180 μl of water (Milli-Q) and 300 ml N-hexane (CAS nr: 110-54-3). The solution was mixed while stirred and heated (55° C.) until viscous solution was obtained.

The solution was dried in a spray drier of the mark SDMicro™ of GEA Niro A/S, using a mass flow of 1000 g/h, a solution temperature of 600° C. and nitrogen as drying gas, whereby the ingoing temperature was 50° C., and the outgoing was 39° C.

The particles was tested in a standard impactor(5-step MLI, Multistage Liquid Impinger). At the test condition (30 l/min) the cut-off values for step 3 and 4 is 4.38 μm and 2.40 μm. The two first steps (1 and 2) contained water (20 ml) to avoid particle bouncing.

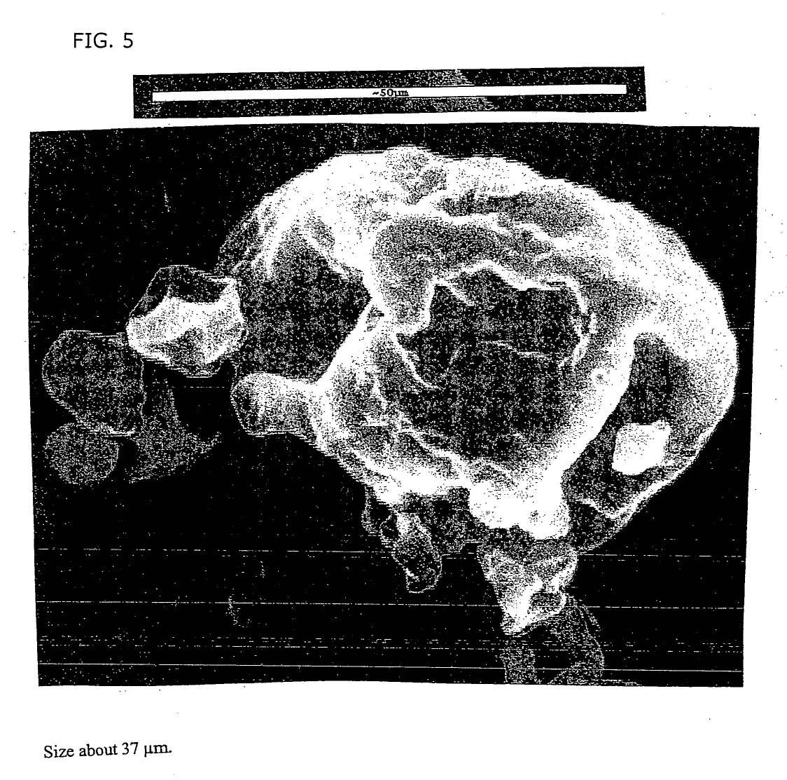

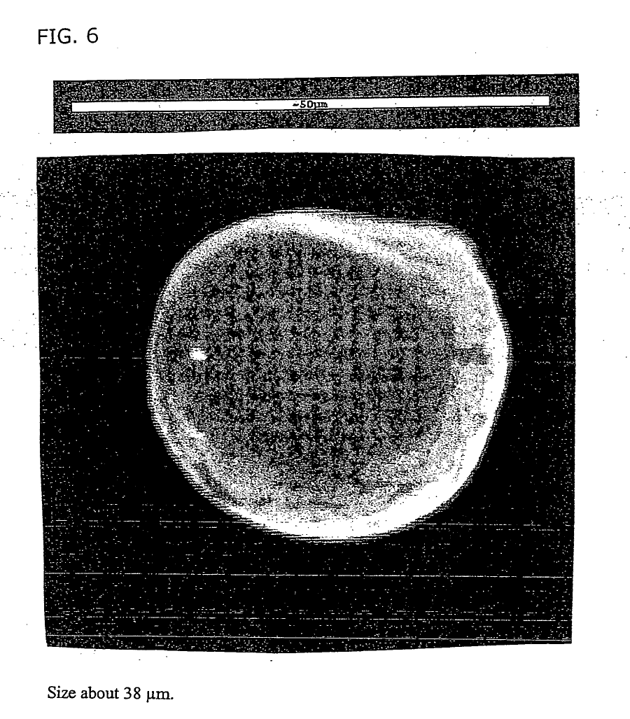

In the accompanying photographs, SEM, particles are shown which have attached to step 4 (FIG. 5,6). The particles on the photographs have a geometrical diameter that is about 10-times or more, larger than the aerodynamic diameter that normally get caught on this step (2,4 μm).

### EXAMPLE 4

A solution consisting of 0.9 g of natural surfactant extract of Porcine origin was mixed with 550 μl of water (Milli-Q) and 90 ml N-hexane (CAS nr: 110-54-3).

The solution was dried in a spray drier of the mark SDMicro™ of GEA Niro A/S, using a mass flow of 900 g/h, a solution temperature of 60° C. and nitrogen as drying gas, whereby the ingoing temperature was 50° C., and the outgoing was 40° C.

The particles was tested in a standard impactor (5-step ML, Multistage Liquid Impinger). At the test condition (30 l/min) the cut-off values for step 3 and 4 is 4.38 μm and 2.40 μm. The two first steps (1 and 2) contained water (20 ml) to avoid particle bouncing.

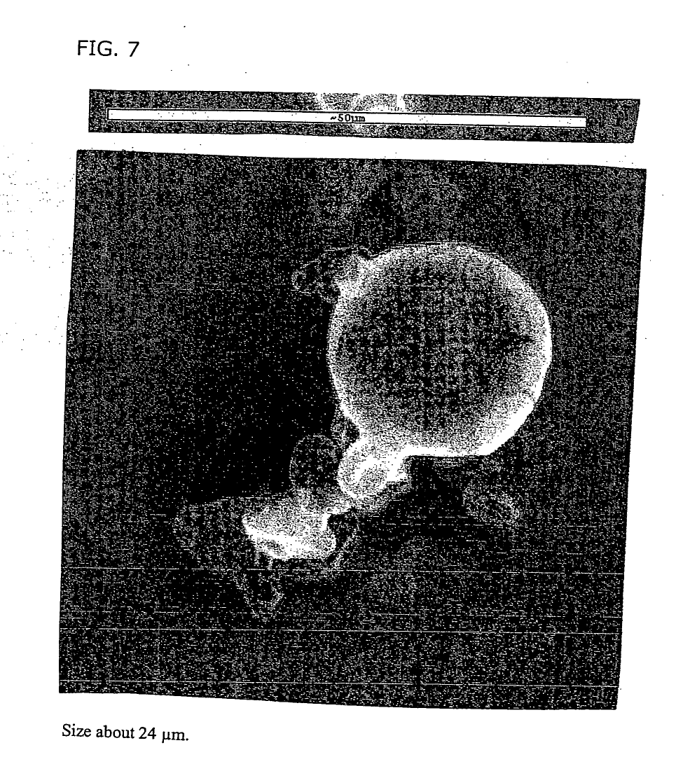

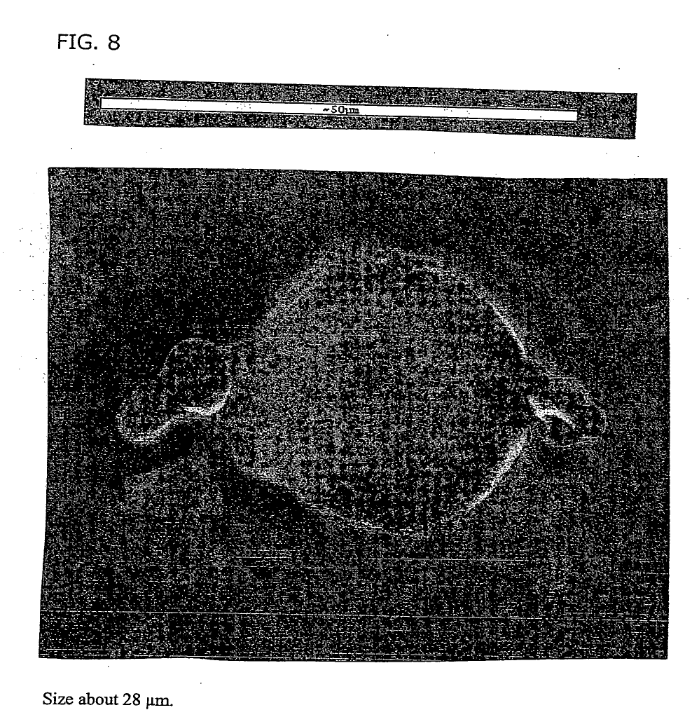

In the accompanying photographs, SEM, particles are shown which have attached to step 4 (FIG. 7-8).

The particles on the photographs have a geometrical diameter that is 10 times or more, larger than the aerodynamic diameter that normally get caught on theses steps (2.4 μm).

### EXAMPLE 5

The powder from example 3 was mixed with dry latex particles to test the lipid particles as carrier particles.

Latex spheres 7 μm, density 1.05 g/cm³, DC-07 from Duke Scientific Corporation. Lipid powder (0.0114 g) and latex spheres (0.0099 g) was mixed in a vial using a Vortex genie 2 from scientific industries.

The powder was tested in a standard impactor (5-step MLI, Multistage Liquid Impinger). At the test condition (30 l/min) the cut-off values for step 3 and 4 is 4.38 μm and 2.40 μm. The two first steps (1 and 2) contained water (20 ml) to avoid particle bouncing.

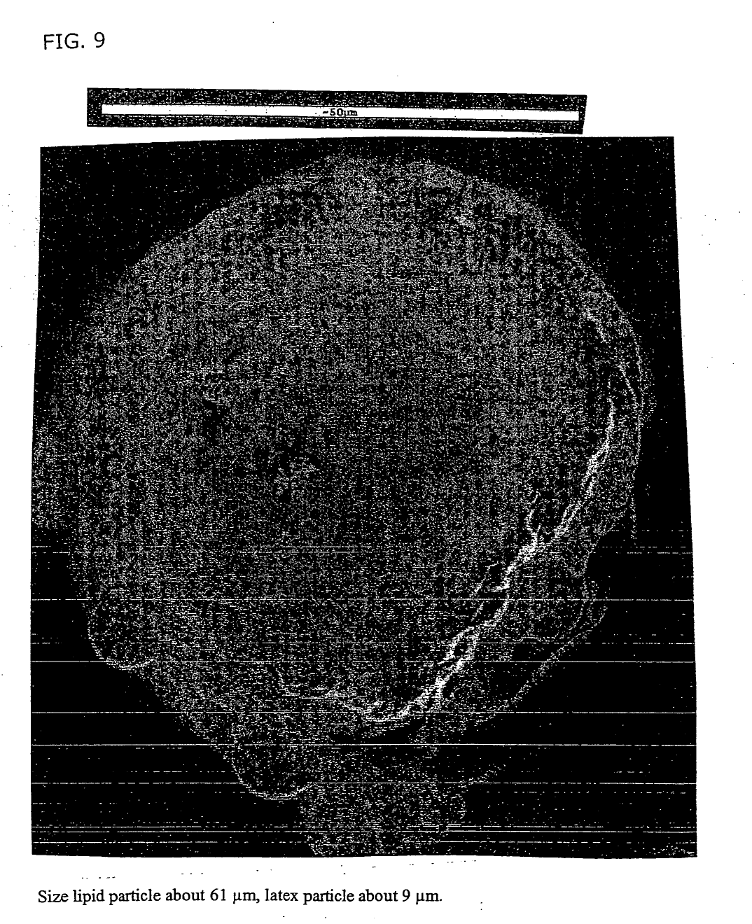

In the accompanying photographs, SEM, particles are shown which have attached to step 3 (FIG. 9).

The large lipid particle on the photograph have a geometrical diameter that is 10 times or more, larger than the aerodynamic diameter that normally get caught on this step. The smaller latex particles attached on the surface have a geometrical diameter that is 2 times or more, larger than the aerodynamic diameter that normally get caught on this step (4.38 μm). This shows that these particles may serve as vehicle for non-Inhalable particles.

### EXAMPLE 6

A solution consisting of 1 g of natural lung surfactant extracts (Curosurf®) mixed with 200 μl of water (Milli-Q) and 100 ml N-hexane (CAS nr: 110-54-3). The solution was mixed while stirred and heated (55° C.) until a viscous solution was obtained. The solution was dried in a spray drier (of the mark SDMicro™ of GEA Niro A/S, using a mass flow of 1000 g/h, a solution temperature of 60° C. and nitrogen as drying gas, whereby the ingoing temperature was 50° C., and the outgoing was 39° C.

Fine particle fraction (FPF) determined by MLI, Multistage Liquid Impinger and chemical analysis.

### EXAMPLE 7

A solution consisting of 3 g DPPC (CAS nr: 63-89-8) and 0.15 g Cholesterol (CAS nr:57-88-5) was mixed with 180 μl of water (Milli-Q) and 300 ml N-hexane (CAS nr: 110-54-3). The solution was mixed while stirred and heated (55° C.) until a viscous solution was obtained.

The solution was dried in a spray drier.

Fine particle fraction (FPF) determined by MLI, Multistage Liquid Impinger and chemical analysis.

### EXAMPLE 8

A solution consisting of 1 g sphingomyelin (CAS nr: 85187-10-6) was mixed with 60 μl of water (Milli-Q) and 100 ml N-hexane (CAS nr: 110-54-3). The solution was mixed while stirred and heated until a viscous solution was obtained.

The solution was dried in a spray drier.

Fine particle fraction (FPF) determined by MLI, Multistage Liquid Impinger and chemical analysis.

### EXAMPLE 9

A solution consisting of 0.8 g of glycerol dioleat GDO (CAS nr:25637-87-7) and 0.2 g DPPC (CAS nr: 63-89-8) was mixed with 60 μl of water (Mili-Q) and 100 ml N-hexane (CAS nr: 110-54-3). The solution was mixed while stirred and heated until a viscous solution was obtained.

The solution was dried in a spray drier.

Fine particle fraction (FPF) determined by MLI, Multistage Liquid Impinger and chemical analysis.

### EXAMPLE 10

A solution consisting of 1 g of monogalactocyidiacylglycerol (MGDG) was mixed with 60 μl of water (Milil-Q) and 100 ml N-hexane (CAS nr: 110-543). The solution was mixed while stirred and heated until a viscous solution was obtained.

The solution was dried in a spray drier.

Fine particle fraction (FPF) determined by MLI, Multistage Liquid Impinger and chemical analysis.

### EXAMPLE 11

A solution consisting of 1 g of digalactocyldiacylglycerol (DGDG) was mixed with 60 μl of water (Milli-Q) and 100 ml N-hexane (CAS nr: 110-543). The solution was mixed while stirred and heated until a viscous solution was obtained.

The solution was dried in a spray drier.

Fine particle fraction (FPF) determined by MLI, Multistage Liquid Impinger and chemical analysis.

### EXAMPLE 12

A solution consisting of 2 g of DMPC(CAS nr:18194-24-6), 1 g DPPC (CAS nr: 63-89-8) and 0.3 g cyclosporine (CAS nr 79217-60-0) was mixed with 180 μl of water (Milli-Q) and 300 ml N-hexane (CAS nr: 110-54-3). The solution was mixed while stirred and heated (55° C.) until a viscous solution was obtained.

The solution was dried in a spray drier.

Fine particle fraction (FPF) determined by MLI, Multistage Liquid Impinger and chemical analysis.

### EXAMPLE 13

A solution consisting of 2 g of DMPC (CAS nr: 18194-24-6), 1 g DPPC(CAS nr: 63-89-8) and 0.015 g calcitonin was mixed with 180 μl of water (Milli-Q) and 300 ml N-hexane (CAS nr: 110-54-3). The solution was mixed while stirred and heated (55° C.) until a viscous solution was obtained.

The solution was dried in a spray drier.

Fine particle fraction (FPF) determined by MLI, Multistage Liquid Impinger and chemical analysis.

### EXAMPLE 14

A solution consisting of 2 g of DMPC (CAS nr:18194-24-6), 1 g DPPC(CAS nr: 63-89-8) and 0.015 g formoterol (CAS nr: 73573-87-2) was mixed with 180 μl of water (Milli-Q) and 300 ml N-hexane (CAS nr: 110-54-3). The solution was mixed while stirred and heated (55° C.) until a viscous solution was obtained.

The solution was dried in a spray drier.

Fine particle fraction (FPF) determined by MLI, Multistage Liquid Impinger and chemical analysis.

### EXAMPLE 15

Powder made according to example 3 was mixed with dry particles of formoterol (CAS nr: 73573-87-2). Lipid powder (0.01 g) and formoterol particles (0.005 g) was mixed in a vial using a Vortex genie 2 from Scientific Industries.

Fine particle fraction (FPF) determined by MLI, Multistage Liquid Impinger and chemical analysis.

### EXAMPLE 16

Powder made according to example 3 was mixed with dry particles of budesonide (CAS nr: 51333-22-3). Lipid powder (0.01 g) and budesonide particles (0.01 g) was mixed in a vial using a Vortex genie 2 from Scientific Industries.

Fine particle fraction (FPF) determined by MLI, Multistage Liquid Impinger and chemical analysis.

### EXAMPLE 17

Powder made according to example 3 was mixed with dry particles of cyclosporin (CAS nr 79217-60-0). Lipid powder (0.01 g) and cyclosporin (0.005 g) was mixed in a vial using a Vortex genie 2 from Scientific Industries.

Fine particle fraction (FPF) determined by MLI, Multistage Liquid Impinger and chemical analysis.

### EXAMPLE 18

Powder made according to example 3 was mixed with dry particles of calcitonin. Lipid powder (0.01 g) and calcitonin (0.01 g) was mixed in a vial using a Vortex genie 2 from Scientific Industries.

Fine particle fraction (FPF) determined by MLI, Multistage Liquid Impinger and chemical analysis.

### REFERENCES

Bhat, M. G., G. W. Cuff, et al. (2000). WO 01/93837 A2
Dennis, et. al. (1993). Biochemistry 32: 10185.
Edwards, D. A., G. Caponetti, et al. (1999). U.S. Pat. No. 5,874,064.
Edwards, D. A., G. Caponetti, et al. (2001). U.S. Pat. No. 6,254,854
Edwards, D. A., 3. Hanes, et al. (1997). “Large Porous Particles for Pulmonary Drug Delivery.” Science 276: 1868-1871.
Edwards, D. A., R. S. Langer, et al. (1999). U.S. Pat. No. 5,985,309
Etter, Jeffrey B. (2000) WO 01/45731 A1
Gunstone, F. D., J. L. Harwood, et al. (1986). The lipid handbook. London New York, Chapman and Hall.
Hanes, J., D. A. Edwards, et al. (1999). U.S. Pat. No. 5,855,913
Harwigsson, I. (1995). Surfactant aggregation and its application to drag reduction. Physical chemistry 1. Lund, Lund University.
Hills, A. B., D. A. Woodcock, et al. (2000). WO0030654
Hinds, W. C. (1982). Aerosol Technology. New York, John Wiley & Sons, Inc.
Hoffmann, H. and G. Ebert (1988). “Surfactants, Micelles and Fascinating Phenomena.” Angew. Chem. Int. Ed. Engl. 27: 902-912.
Hollis, G. L., Ed. (1995). SURFACTANTS EUROPA.
Israelachvill, J. N. (1992). Intermolecular & Surface Forces. San Diego, Academic press Limited.

Israelachvili, J. N., D. J. Mitchell, et al. (1976). J. Chem. Soc. Faraday Trans. 2: 1525.

Klein, D. H., L. A. Trevino, et al. (1996). WO9626746
Mitchell, D. J., G. J. T. Tiddy, et al. (1983). J. Chem. Soc. Faraday Trans. 79: 975.
Morgan, S. E. and C. L. McCormick (1990). Prog. Polym. Sci. 15: 507.
Ogden, J. (1996). Scrip Magazine: 56.
Pruss, T. P. (1997). U.S. Pat. No. 5,698,537
Reul, B. and W. Petri (1997). U.S. Pat. No. 5,663,198
Riess, 3. G., E. G. Schutt, et al. (2001). WO0185136
Scartazzini, R. and P. L. Lulsi (1988). “Organogels from Lecithins.” J. Phys. Chem. 92: 829-833.
Stanforth, J. N. (1996). US006153224A
Tanford, C. (1973). The hydrofobic effect: formulation of micelles and biological membranes., John Wiley &Sons Inc.
Tarara, T. E., J. G. Weers, et al. (2000). WO9916419
Tartar, H. V. (1955). J. Phys. Chem. 59: 1195.
Tsai, e. a. (1988). Biochemistry 27: 4619.
Vanbever, R., 3. D. Mintzes, et al. (1999). “Formulation and Physical Characterization of Large Porous Particles for Inhalation.” Pharmaceutical Research 16(11): 1735.
Weers, J. G., T. E. Tarara, et al. (2001). U.S. 2002/00172 95 A1.

1. Pharmaceutical, porous particles having a density of <0.5 g/cm³ to be used in therapeutical applications, optionally comprising one or more therapeutically active compound(-s) or substance(-s), whereby the particles consist of one or more network forming compounds, which in diluted solutions self associate to large three dimensional structures.

2. Pharmaceutical particles according to claim 1, wherein the particle comprises one or more lipid(-s) forming a three dimensional structure.

3. Pharmaceutical particles according to claim 2, wherein the particles having the density of 0.001 to 0.5 g/cm³, and an aerodynamic diameter (dₐ) if 0.1 to 10 μm comprise one or more phospholipids(-s) forming a three dimensional structure.

4. Pharmaceutical particles according to claim 3, wherein the phospholipids is a constituent bodily phospholipid.

5. Pharmaceutical particles according to claim 1, wherein the particles forming a three dimensional structure are free flowing.

6. Pharmaceutical particles according to claim 3, wherein the geometrical diameter (d_{g}) is at least 3 μm.

7. Pharmaceutical particles according to claim 3, wherein the geometrical diameter is 10 to 30 μm.

8. Pharmaceutical particles according to claim 3, wherein the geometrical diameter is >30 μm.

9. Pharmaceutical particles according to claim 3, wherein the aerodynamic diameter (dₐ) is 0.5 to 5 μm.

10. Pharmaceutical particles according to claim 9, wherein the aerodynamic diameter (dₐ) is 1 to 5 μm.

11. Pharmaceutical particles according to claim 1, wherein the particle comprises one or more therapeutically active compound(-s) or substance(-s) molecularly bound to the three dimensional structure.

12. Pharmaceutical particles according to claim 1, wherein the particle comprises one or more therapeutically active compound(-s) or substance(-s) adhered to the three dimensional structure.

13. Pharmaceutical particles according to claim 1, wherein the particle comprises one or more therapeutically active compound(-s) or substance(-s) of the group of diagnostic agents, prophylactically active pharmaceuticals, therapeutically active pharmaceuticals, and immunizing agents.

14. Pharmaceutical particles according to claim 13, wherein the drug(-s) is selected form the group consisting of antiallergics, analgesics, bronchodilators, antihistamines, antiviral agents, antibiotics, anti-inflammatories, immunomodulators, antidiabetics, peptides, and steroids.

15. Pharmaceutical particles according to claim 14, wherein the particle comprises one or more therapeutically active compound(-s) or substance(-s) selected from the group consisting of adrenaline, albuterol, atropine, beclomethasone dipropionate, budesonide, butixocort propionate, clemastine, cromolyn, epinephrine, ephedrine, fentanyl, flunisolide, fluticasone, formoterol, ipratropium bromide, isoproterenol, lidocaine, morphine, nedocromil, pentamidine isoethionate, pirbuterol, prednisolone, salmeterol, terbutaline, tetracycline, 4-amino-.alpha.,.alpha.,2-trimethyl-1H-imadaxo[4,5-c]quinoline-1-ethanol, 2,5-diethyl-10-oxo-1,2,4-triazolo[1,5-c]pyrimido[5,4-b][1,4]thiazine, 1-(1-ethylpropyl)-1-hydroxy-3-phenylurea, insulin and pharmaceutically acceptable salts and solvates thereof, and mixtures thereof.

16. Pharmaceutical particles according to claim 1, wherein the particles are intended for inhalation use.

17. Pharmaceutical particles according to claim 1, wherein the particle is intended for topical use.

18. A process for the manufacture of an inhalable particles having a density of <0.5 g/cm³ according to claim 1, having an aerodynamic diameter (dₐ) of at least 0.5 μm, whereby a solution of a one or more network forming compounds, which in diluted solutions self associates to large three dimensional structures is evaporated to dryness.

19. A process for the manufacture of inhalable particles according to claim 18, whereby the solution comprises up to 10% dry matter, preferably up to 1% dry matter before being evaporated to dryness.

20. A process according to claim 18, wherein between 1 and 30 moles of water per mole of structure forming material is present.

21. A process according to claim 19, wherein the amount of water is 1-20 moles per mole of three dimensional structure forming material to form a reversed structure.

22. A process according to claim 21, wherein the amount of water is 1-10 moles per mole of three dimensional structure forming material.

23. A process according to claim 19, wherein water is present in an excess of three dimensional structure forming material to form a straight structure wherein the aqueous phase in continuous.

24. Pharmaceutical particles according to claim 16, wherein the particles are suitable for use as carrier particles, preferably in ordered mixtures, capable of forming inhalable aggregates having an aerodynamic diameter (dₐ) of 0.5 to 5 μm and consisting of a substantial amount of other particles.

25. Pharmaceutical particle according to claim 24, wherein the other particles are drug particles having a density of 0.5 g/cm³ or greater, a high density drug particles.

26. A method for medical treatment of human or animal body comprising administering pharmaceutical particles as claimed in claim 1.

27. A method according to claim 26, wherein said particles are administered by inhalation.

28. Particles as claimed in claim 1, for use in therapy.

29. The use of particles as claimed in claim 1, in the manufacture of a medicament for use in therapy.
